(19) United States
(12) Patent Application Publication
(10) Pub. No.: US 2025/0152508 A1
(43) Pub. Date: May 15, 2025

(54) SALBUTAMOL DELIVERY COMPOSITIONS, DEVICES AND METHODS

(71) Applicant: HONEYWELL INTERNATIONAL INC., Charlotte, NC (US)

(72) Inventors: Erik Boldt, Charlotte, NC (US); Gregory Smith, Charlotte, NC (US)

(73) Assignee: HONEYWELL INTERNATIONAL INC., Charlotte, NC (US)

(21) Appl. No.: 18/939,379

(22) Filed: Nov. 6, 2024

Related U.S. Application Data
(60) Provisional application No. 63/548,215, filed on Nov. 12, 2023.

(51) Int. Cl. A61K 9/00; A61K 9/12; A61K 31/137; A61K 47/06; A61K 47/10; A61K 47/12

(52) U.S. Cl. CPC A61K 9/008; A61K 9/124; A61K 31/137; A61K 47/06; A61K 47/10; A61K 47/12

(57) ABSTRACT

Disclosed are medicinal compositions, and devices, methods and systems which use same, comprising a carrier and at least one medicinally active compound, said carrier comprising at least 1234ze(E) and ethanol.

### FIELD OF THE INVENTION

This invention relates to medicament delivery compositions, systems, devices and methods. In particular aspects, this invention relates to medicinal aerosol compositions, methods and devices used for metered dose delivery of salbutamol.

### BACKGROUND OF THE INVENTION

Metered dose inhalers (MDIs) have long been used to deliver medicaments, such as bronchodilator drugs and steroids, to the areas of patients needing treatment. Compared with oral administration of bronchodilators, inhalation therapy using MDIs frequently has the advantage of relatively rapid onset of action and relatively low instance of systemic side effects.

MDIs may be used to deliver medicaments in a solubilized form or as a suspension. Typically, MDIs use a relatively high vapor pressure propellant to carry and expel aerosolized droplets containing an API into the respiratory tract when the MDI is activated. The propellant/carrier for the active pharmaceutical ingredient (sometimes referred to herein as “API”) must be safe for patients' use and be pharmaceutically acceptable. The API to be delivered by an MDI is typically provided as a suspension of particulates dispersed within a carrier which helps to form a suspension and/or otherwise carry the active ingredient.

Several challenges exist in connection with efforts to provide new MDIs in general, and suspension-based MDI propellants in particular. For example, while there are advantages to delivery of the API as fine particles suspended in a propellant, there is a tendency of such particles of API to aggregate and/or flocculate. This problem can have an impact on the effectiveness of use of the MDI. In a typical case, the user will shake the MDI device in anticipation of use in order to establish the suspension. However, in some cases the length of time that the suspension is maintained without flocculation and/or aggregation can be undesirably short, potentially negatively impacting the efficacy of delivering the active ingredient as intended. One factor which influences the tendency of flocculation and/or agglomeration to occur is the amount of the API in the suspension, with increasing amounts of API tending to aggravate the flocculation/agglomeration problem. Thus, it has been heretofore most common to overcome this problem by requiring the user to active the MDI more than once (usually twice) in order to introduce the desired dosage to the target area, thus permitting the amount of active ingredient contained in the carrier to be lower. For example, by requiring the user to activate the MDI twice to deliver the desired dosage, the amount of active ingredient suspended in the carrier can be reduced by 50%, thus reducing the flocculation/aggregation problem.

Applicants have come to appreciate that requiring more than one activation of the MDI to deliver the desired dose can have detrimental consequences. For example, it is possible that for prior systems and methods only half of the desired dosage will be delivered if the user does not carefully read the instructions for use and/or forgets the instructions and only activates the MDI once. Thus, one object of the present invention is the development of a carrier that is able to avoid flocculation/aggregation problems, and other MDI problems, while at the same time providing an increased concentration of active ingredient carried in suspension. Achieving this result is a significant challenge because of the myriad of performance properties that at once must be achieved with relatively high concentrations of active ingredients.

Carrier properties which can have an impact on MDI performance can include appropriate boiling point and vapor pressure so that it can be liquefied in a closed container at room temperature but develop a high enough pressure when the MDI is activated to deliver the drug as an atomized composition even at low ambient temperatures. Further, the carrier should be of low acute and chronic toxicity. It should have a high degree of chemical stability in contact with the drug, the container and the metallic and non-metallic components of the MDI device, and have a low propensity to extract low molecular weight substances from any elastomeric materials in the MDI device. The carrier preferably also is able to maintain the drug, including relatively high concentrations of drug, in a stable suspension or in a stable dispersion for a sufficient time to permit reproducible delivery of the drug in use. When the drug is in suspension in the carrier, the density of the liquid carrier is desirably similar to that of the solid drug in order to avoid rapid sinking or floating of the drug particles in the liquid. Finally, the carrier should not present a significant flammability risk to the patient in use. In particular, it should form a non-flammable or low flammability mixture when mixed with air in the respiratory tract. Environmentally desirable properties, such as low GWP and low ODP, are also generally highly desirable.

U.S. Pat. No. 9,308,199, which is assigned to the assignee of the present application, describes the use of fluoroolefins, preferably hydrofluorolefins (HFO), as medicinally acceptable carriers that are able to overcome the environmental deficiencies of CFCs, HFCs and HCFCs mentioned above. Tetrafluoropropenes, including 1,3,3,3-tetrafluoropropene (HFO-1234ze) and 2,3,3,3-tetrafluoropropene (HFO-1234yf) are disclosed as being preferred.

WO2023/039103 mentions that HFOs have been proposed as propellants for MDIs but also notes that no MDI product has been successfully developed or commercialized using HFOs as a propellant. The ′103 publication discloses an MDI that uses a formulation comprising greater than 70% by weight of HFO-1234ze(E), ethanol and at least one active pharmaceutical ingredient (API). The amounts of ethanol disclosed as being used in the ′103 formulations range from as low as 0.1 wt. % to as high as 20%.

Asthma is one condition that has frequently been treated by the use of MDIs. Asthma has been described as a chronic disease that involves inflammation of the pulmonary airways and bronchial hyperresponsiveness that results in the clinical expression of a lower airway obstruction that usually is reversible. The pathophysiology of asthma or related disorders involves bronchoconstriction resulting from bronchial smooth muscle spasm and airway inflammation with mucosal edema. Treatment of asthma and other related disorders (including Chronic Obstructive Pulmonary Disease (COPD)) has included the administration of β-2 agonists, also known as, β-2 adrenoreceptor agonists. Such β-2 adrenoreceptor agonists are known to provide a bronchodilator effect to patients, resulting in relief from the symptoms of breathlessness. More particularly, β-2 adrenoreceptor agonists have been shown to increase the conductance of potassium channels in airway muscle cells, leading to membrane hyperpolarization and relaxation. Salbutamol is a short-acting β-2 adrenoreceptor and has been recommended and used for the relief of acute asthma symptoms.

Notwithstanding the disclosures as mentioned above, applicants have come to recognize the need for delivery compositions, systems, devices and methods for salbutamol that at once provide relatively low ozone depletion potential, relatively low global warming potential and the ability to maintaining the API, especially in relatively high concentrations, in a stable suspension or in a stable dispersion for a sufficient time to permit reproducible accurate delivery of the drug in use.

### SUMMARY

Applicants have found that many of the shortcomings of the prior compositions can be overcome and/or that many of the above-noted needs can be satisfied by pharmaceutical compositions of the present invention and the use thereof in MDIs and inhalation delivery methods.

The present invention includes pharmaceutical compositions comprising:
a. fine particles of salbutamol sulfate;
b. a carrier in which said fine particles of salbutamol sulfate are suspended, said carrier comprising: (i) from above about 96% by weight to less than 98% by weight of HFO-1234ze(E); and (ii) from greater than 2% by weight to less than 4% by weight of ethanol.
   For the purposes of convenience, pharmaceutical compositions according to this paragraph are referred to as Pharmaceutical Composition 1A.

The present invention includes pharmaceutical compositions consisting essentially of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); and (ii) from greater than 2% by weight to less than 4% by weight of ethanol.
   For the purposes of convenience, pharmaceutical compositions according to this paragraph are referred to as Pharmaceutical Composition 1B.

The present invention includes pharmaceutical compositions consisting of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and (iii) optionally one or more excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 1C.

The present invention includes pharmaceutical compositions comprising:
a. fine particles of salbutamol;
b. a carrier in which said fine particles of salbutamol are suspended, said carrier comprising: (i) from above about 96% by weight to less than 98% by weight of HFO-1234ze(E); and (ii) from greater than 2% by weight to less than 4% by weight of ethanol.
   For the purposes of convenience, pharmaceutical compositions according to this paragraph are referred to as Pharmaceutical Composition 1D.

The present invention includes pharmaceutical compositions consisting essentially of:
a. fine particles of salbutamol;
b. a carrier in which said fine particles of salbutamol are suspended, said carrier comprising: (i) from above about 96% by weight to less than 98% by weight of HFO-1234ze(E); (ii) from greater than 2% by weight to less than 4% by weight of ethanol; and (iii) oleic acid.
   For the purposes of convenience, pharmaceutical compositions according to this paragraph are referred to as Pharmaceutical Composition 1 E.

The present invention includes pharmaceutical compositions consisting of:
a. fine particles of salbutamol;
b. a carrier in which said fine particles of salbutamol are suspended, said carrier comprising: (i) from above about 96% by weight to less than 98% by weight of HFO-1234ze(E); and (ii) from greater than 2% by weight to less than 4% by weight of ethanol.
   For the purposes of convenience, pharmaceutical compositions according to this paragraph are referred to as Pharmaceutical Composition 1F.

The present invention includes pharmaceutical compositions comprising:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and (iii) oleic acid.
   For the purposes of convenience, pharmaceutical compositions according to this paragraph are referred to as Pharmaceutical Composition 2A.

The present invention includes pharmaceutical compositions consisting essentially of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and (iii) oleic acid.
   For the purposes of convenience, pharmaceutical compositions according to this paragraph are referred to as Pharmaceutical Composition 2B.

The present invention includes pharmaceutical compositions consisting of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and (iii) at least oleic acid and optionally one or more additional excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 2C.

The present invention includes pharmaceutical compositions comprising:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and from greater than 0.005% by weight to 0.015% by weight of oleic acid.
   For the purposes of convenience, pharmaceutical compositions according to this paragraph are referred to as Pharmaceutical Composition 2D.

The present invention includes pharmaceutical compositions consisting essentially of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and from greater than 0.005% by weight to 0.015% by weight of oleic acid.
   For the purposes of convenience, pharmaceutical compositions according to this paragraph are referred to as Pharmaceutical Composition 2E.

The present invention includes pharmaceutical compositions consisting of:
a. fine particles of salbutamol sulfate;
b. a carrier in which said fine particles of salbutamol sulfate are suspended, said carrier comprising: (i) from above about 96% by weight to less than 98% by weight of HFO-1234ze(E); (ii) from greater than 2% by weight to less than 4% by weight of ethanol; and from greater than 0.005% by weight to 0.015% by weight of oleic acid and optionally one or more additional excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 2F.

The present invention includes pharmaceutical compositions comprising:
a. fine particles of salbutamol;
b. a carrier in which said fine particles of salbutamol are suspended, said carrier comprising: (i) from above about 96% by weight to less than 98% by weight of HFO-1234ze(E); (ii) from greater than 2% by weight to less than 4% by weight of ethanol; and (iii) oleic acid.
   For the purposes of convenience, pharmaceutical compositions according to this paragraph are referred to as Pharmaceutical Composition 2G.

The present invention includes pharmaceutical compositions comprising:
a. from about 2.5 mg/mL to less than about 4.5 mg/mL of fine particles of salbutamol sulfate;
b. a carrier in which said fine particles of salbutamol sulfate are suspended, said carrier comprising: (i) from greater than about 95% by weight to less than 99% by weight of HFO-1234ze(E); and (ii) from greater than 1% by weight to less than about 5% by weight of ethanol. For the purposes of convenience, pharmaceutical compositions according to this paragraph are referred to as Pharmaceutical Composition 3A.

The present invention includes pharmaceutical compositions consisting essentially of:
a. from about 2.5 mg/mL to less than about 4.5 mg/mL of fine particles of salbutamol sulfate;
b. a carrier in which said fine particles of salbutamol sulfate are suspended, said carrier comprising: (i) from greater than about 95% by weight to less than 99% by weight of HFO-1234ze(E); and (ii) from greater than 1% by weight to less than about 5% by weight of ethanol.
   For the purposes of convenience, pharmaceutical compositions according to this paragraph are referred to as Pharmaceutical Composition 3B.

The present invention includes pharmaceutical compositions consisting of:
a. from about 2.5 mg/mL to about 4.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) optionally one or more excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 3C.

The present invention includes pharmaceutical compositions comprising:
a. from about 2.5 mg/mL to about 4.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015% by weight of oleic acid.
   For the purposes of convenience, pharmaceutical compositions according to this paragraph are referred to as Pharmaceutical Composition 3D.

The present invention includes pharmaceutical compositions consisting essentially of:
a. from about 2.5 mg/mL to about 4.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015% by weight of oleic acid.
   For the purposes of convenience, pharmaceutical compositions according to this paragraph are referred to as Pharmaceutical Composition 3E.

The present invention includes pharmaceutical compositions consisting of:
a. from about 2.5 mg/mL to about 4.5 mg/mL of fine particles of salbutamol sulfate;
b. a carrier in which fine particles of salbutamol sulfate suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015% by weight of oleic acid and optionally one or more additional excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 3F.

The present invention includes pharmaceutical compositions comprising:
a. from about 2.5 mg/mL to less than about 4.5 mg/mL of fine particles of salbutamol;
b. a carrier in which said fine particles of salbutamol are suspended, said carrier comprising: (i) from greater than about 95% by weight to less than 99% by weight of HFO-1234ze(E); and (ii) from greater than 1% by weight to less than about 5% by weight of ethanol.
   For the purposes of convenience, pharmaceutical compositions according to this paragraph are referred to as Pharmaceutical Composition 3G.

The present invention includes pharmaceutical compositions comprising:
a. from about 2.5 mg/mL to about 3.5 mg/mL of fine particles of salbutamol sulfate;
b. a carrier in which said fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) oleic acid.
   For the purposes of convenience, pharmaceutical compositions according to this paragraph are referred to as Pharmaceutical Composition 4A.

The present invention includes pharmaceutical compositions consisting essentially of:
a. from about 2.5 mg/mL to about 3.5 mg/mL of fine particles of salbutamol sulfate;
b. a carrier in which said fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) oleic acid.
   For the purposes of convenience, pharmaceutical compositions according to this paragraph are referred to as Pharmaceutical Composition 4B.

The present invention includes pharmaceutical compositions consisting of:
a. from about 2.5 mg/mL to about 3.5 mg/mL of fine particles salbutamol sulfate;
b. a carrier in which said fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) oleic acid and optionally one or more additional excipients selected from the group consisting of suspension formation aids, suspension stabilizers, Salbutamol stabilizers and combinations of two or more of these.
   For the purposes of convenience, pharmaceutical compositions according to this paragraph are referred to as Pharmaceutical Composition 4C.

The present invention includes pharmaceutical compositions comprising:
a. from about 2.5 mg/mL to about 3.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015% by weight of oleic acid.
   For the purposes of convenience, pharmaceutical compositions according to this paragraph are referred to as Pharmaceutical Composition 4D.

The present invention includes pharmaceutical compositions consisting essentially of:
a. from about 2.5 mg/mL to about 3.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015 % by weight of oleic acid.
   For the purposes of convenience, pharmaceutical compositions according to this paragraph are referred to as Pharmaceutical Composition 4E.

The present invention includes pharmaceutical compositions consisting of:
a. from about 2.5 mg/mL to about 3.5 mg/mL of fine particles of salbutamol sulfate;
b. a carrier in which fine particles of salbutamol sulfate are suspended, said carrier comprising: (i) from greater than about 95% by weight to less than 99% by weight of HFO-1234ze(E); (ii) from greater than 1% by weight to less than about 5% by weight of ethanol; and (iii) and from greater than 0.005% by weight to 0.015% by weight of oleic acid and optionally one or more additional excipients selected from the group consisting of suspension formation aids, suspension stabilizers, salbutamol stabilizers and combinations of two or more of these.
   For the purposes of convenience, pharmaceutical compositions according to this paragraph are referred to as Pharmaceutical Composition 4F.

The present invention includes pharmaceutical compositions comprising:
a. from about 2.5 mg/mL to about 3.5 mg/mL of fine particles of salbutamol;
b. a carrier in which said fine particles of salbutamol are suspended, said carrier comprising: (i) from greater than about 95% by weight to less than 99% by weight of HFO-1234ze(E); (ii) from greater than 1% by weight to less than about 5% by weight of ethanol; and (iii) oleic acid.
   For the purposes of convenience, pharmaceutical compositions according to this paragraph are referred to as Pharmaceutical Composition 4G.

The present invention includes pharmaceutical compositions in the form of an aerosol comprising:
a. fine particles of salbutamol; and
b. a carrier carrying said fine particles of salbutamol and comprising: (i) from above about 96% by weight to less than 98% by weight of HFO-1234ze(E); and (ii) from greater than 1% by weight to less than 4% by weight of ethanol, wherein the amount of said salbutamol in said aerosol is from about greater than 150 micrograms to about 300 micrograms.
   For the purposes of convenience, pharmaceutical compositions according to this paragraph are referred to as Pharmaceutical Composition 5A.

The present invention includes pharmaceutical compositions in the form of an aerosol consisting essentially of:
a. fine particles of salbutamol sulfate; and
b. a carrier carrying said fine particles of salbutamol sulfate and comprising: (i) from above about 96% by weight to less than 98% by weight of HFO-1234ze(E); and (ii) from greater than 1% by weight to less than 4% by weight of ethanol, wherein the amount of said salbutamol sulfate in said aerosol is from about greater than 150 micrograms to about 300 micrograms.
   For the purposes of convenience, pharmaceutical compositions according to this paragraph are referred to as Pharmaceutical Composition 5B.

The present invention includes pharmaceutical compositions in the form of an aerosol consisting of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) optionally one or more excipients selected from the group consisting of suspension formation aids, suspension stabilizers, salbutamol stabilizers and combinations of two or more of these, wherein the amount of said salbutamol sulfate in said aerosol is from about greater than 150 micrograms to about 300 micrograms.
   For the purposes of convenience, pharmaceutical compositions according to this paragraph are referred to as Pharmaceutical Composition 5C.

The present invention includes pharmaceutical compositions in the form of an aerosol comprising:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.015% by weight of oleic acid, wherein the amount of said salbutamol sulfate in said aerosol is from about greater than 150 micrograms to about 300 micrograms.
   For the purposes of convenience, pharmaceutical compositions according to this paragraph are referred to as Pharmaceutical Composition 5D.

The present invention includes pharmaceutical compositions in the form of an aerosol consisting essentially of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.015% by weight of oleic acid, wherein the amount of said salbutamol sulfate in said aerosol is from about greater than 150 micrograms to about 300 micrograms.
   For the purposes of convenience, pharmaceutical compositions according to this paragraph are referred to as Pharmaceutical Composition 5E.

The present invention includes pharmaceutical compositions in the form of an aerosol consisting of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.015% by weight of oleic acid and optionally one or more excipients selected from the group consisting of suspension formation aids, suspension stabilizers, salbutamol stabilizers and combinations of two or more of these, wherein the amount of said salbutamol sulfate in said aerosol is from about greater than 150 micrograms to about 300 micrograms.
   For the purposes of convenience, pharmaceutical compositions according to this paragraph are referred to as Pharmaceutical Composition 5F.

The present invention includes pharmaceutical compositions in the form of an aerosol comprising:
a. fine particles of salbutamol; and
b. a carrier carrying said fine particles of salbutamol and comprising: (i) from above about 96% by weight to less than 98% by weight of HFO-1234ze(E); and (ii) from greater than 1% by weight to less than 4% by weight of ethanol, wherein the amount of said salbutamol in said aerosol is from about greater than 150 micrograms to about 300 micrograms.
   For the purposes of convenience, pharmaceutical compositions according to this paragraph are referred to as Pharmaceutical Composition 5G.

The present invention includes pharmaceutical compositions in the form of an aerosol comprising:
a. fine particles of salbutamol sulfate; and
b. a carrier carrying said fine particles salbutamol sulfate and comprising: (i) from above about 96% by weight to less than 98% by weight of HFO-1234ze(E); (ii) from greater than 1% by weight to less than 4% by weight of ethanol; and (iii) oleic acid, wherein the amount of said salbutamol sulfate fine particles in said aerosol is from about greater than 150 micrograms to about 300 micrograms.
   For the purposes of convenience, pharmaceutical compositions according to this paragraph are referred to as Pharmaceutical Composition 6A.

The present invention includes pharmaceutical compositions in the form of an aerosol consisting essentially of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) oleic acid, wherein the amount of said salbutamol sulfate fine particles in said aerosol is from about greater than 150 micrograms to about 300 micrograms.
   For the purposes of convenience, pharmaceutical compositions according to this paragraph are referred to as Pharmaceutical Composition 6B.

The present invention includes pharmaceutical compositions in the form of an aerosol consisting of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) oleic acid, wherein the amount of said salbutamol sulfate fine particles in said aerosol is from about greater than 150 micrograms to about 300 micrograms.
   For the purposes of convenience, pharmaceutical compositions according to this paragraph are referred to as Pharmaceutical Composition 6C.

The present invention includes pharmaceutical compositions in the form of an aerosol comprising:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.015% by weight of oleic acid, wherein the amount of said salbutamol sulfate fine particles in said aerosol is from about greater than 150 micrograms to about 300 micrograms.
   For the purposes of convenience, pharmaceutical compositions according to this paragraph are referred to as Pharmaceutical Composition 6D.

The present invention includes pharmaceutical compositions in the form of an aerosol consisting essentially of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.0215% by weight of oleic acid, wherein the amount of said salbutamol sulfate fine particles in said aerosol is from about greater than 150 micrograms to about 300 micrograms.
   For the purposes of convenience, pharmaceutical compositions according to this paragraph are referred to as Pharmaceutical Composition 6E.

The present invention includes pharmaceutical compositions in the form of an aerosol consisting of:
a. fine particles of salbutamol sulfate; and
b. a carrier carrying said fine particles of salbutamol sulfate and comprising: (i) from above about 96% by weight to less than 98% by weight of HFO-1234ze(E); (ii) from greater than 1% by weight to less than 4% by weight of ethanol; and (iii) from greater than 0.005% by weight to 0.015% by weight of and optionally one or more additional excipients selected from the group consisting of suspension formation aids, suspension stabilizers, salbutamol stabilizers and combinations of two or more of these, wherein the amount of said salbutamol sulfate fine particles in said aerosol is from about greater than 150 micrograms to about 300 micrograms. For the purposes of convenience, pharmaceutical compositions according to this paragraph are referred to as Pharmaceutical Composition 6F.

The present invention includes pharmaceutical compositions in the form of an aerosol comprising:
a. fine particles of salbutamol; and
b. a carrier carrying said fine particles of salbutamol and comprising: (i) from above about 96% by weight to less than 98% by weight of HFO-1234ze(E); (ii) from greater than 1% by weight to less than 4% by weight of ethanol; and (iii) oleic acid, wherein the amount of said salbutamol fine particles in said aerosol is from about greater than 150 micrograms to about 300 micrograms.
   For the purposes of convenience, pharmaceutical compositions according to this paragraph are referred to as Pharmaceutical Composition 6G.

The present invention also includes methods for delivering a prescribed dose of salbutamol sulfate comprising:
a. providing in an MDI comprising: (1) a carrier comprising from greater than about 95% by weight to less than 99% by weight of HFO-1234ze(E); and (ii) from greater than 1% by weight to less than about 5% by weight of ethanol; and (2) fine particles of salbutamol sulfate; and
b. actuating said MDI once to produce a single aerosol spray of said salbutamol sulfate, wherein said single spray contains about the prescribed dose of said salbutamol sulfate.
   For the purposes of convenience, methods according to this paragraph are referred to as Pharmaceutical Delivery Method 1A.

The present invention also includes methods for delivering a prescribed dose of salbutamol comprising:
a. providing in an MDI which comprises a pharmaceutical composition of the present invention, including each of Pharmaceutical Composition 1-6; and
b. actuating said MDI once to produce a single aerosol spray of salbutamol fine particles, wherein said single spray contains about the prescribed dose of said salbutamol.
   For the purposes of convenience, methods according to this paragraph are referred to as Pharmaceutical Delivery Method 1B.

The present invention also includes methods for delivering a prescribed dose of salbutamol sulfate comprising:
a. providing in an MDI comprising: (1) a carrier comprising from greater than about 95% by weight to less than 99% by weight of HFO-1234ze(E); and (ii) from greater than 1% by weight to less than about 5% by weight of ethanol; and (2) fine particles of the API carried by said carrier, wherein said API comprises and
b. actuating said MDI once to produce a single aerosol spray of said pharmaceutical composition, wherein said single spray contains said prescribed dose, wherein said prescribed dose is from about 150 micrograms to about 300 micrograms.
   For the purposes of convenience, methods according to this paragraph are referred to as Pharmaceutical Delivery Method 2A.

The present invention also includes methods for delivering a prescribed dose of salbutamol comprising:
a. providing in an MDI which delivers an aerosol spray of a pharmaceutical composition of the present invention, including each of Pharmaceutical Composition 1-6; and
b. actuating said MDI once to produce a single aerosol spray of said pharmaceutical composition, wherein said single spray contains said prescribed dose, wherein said prescribed dose is an amount of from about 150 micrograms to about 300 micrograms of salbutamol.
   For the purposes of convenience, methods according to this paragraph are referred to as Pharmaceutical Delivery Method 2B.

The present invention also includes a metered dose inhaler (MDI) comprising:
a. a container;
b. a pharmaceutical composition under pressure in the container, said pharmaceutical composition comprising: (i) from greater than about 95% by weight to less than 99% by weight of HFO-1234ze(E); (ii) from greater than 1% by weight to less than about 5% by weight of ethanol; and (iii) from about 2.5 mg/mL to about 3.5 mg/mL of salbutamol sulfate, said salbutamol sulfate being in the form of fine particles; and
c. a normally closed valve on the container, said normally closed valve being actuatable to an open position to release an aerosol spray of a predefined volume of said pharmaceutical composition from said container.
   For the purposes of convenience, MDIs according to this paragraph are referred to as MDI 1A.

The present invention also includes a metered dose inhaler (MDI) comprising:
a. a container;
b. a pharmaceutical composition of the present invention, including each of Pharmaceutical Composition 1-6, under pressure in the container; and
c. a normally closed valve on the container, said normally closed valve being actuatable to an open position to release an aerosol spray of a predefined volume of said pharmaceutical composition from said container.
   For the purposes of convenience, MDIs according to this paragraph are referred to as MDI 1B.

The present invention also includes a metered dose inhaler (MDI) comprising:
a. a container;
b. a pharmaceutical composition under pressure in said container, said pharmaceutical composition comprising: (a) a carrier comprising from greater than about 95% by weight to less than 99% by weight of HFO-1234ze(E); and (ii) from greater than 1% by weight to less than about 5% by weight of ethanol; and (b) from about 2.5 mg/mL to about 3.5 mg/mL of fine particles of salbutamol sulfate suspended in said carrier
   For the purposes of convenience, MDIs according to this paragraph are referred to as MDI 2A.

### BRIEF DESCRIPTION OF THE DRAWING

The invention will now be described with reference to the accompanying drawings in which:

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

FIG. 4 is a chart illustrating data from Example 1.

FIG. 5 is a chart illustrating data from Example 1.

FIG. 6 is a chart illustrating data from Example 1.

FIG. 7 is a chart illustrating data from Example 2.

FIG. 8 is a chart illustrating data from Example 2.

FIG. 9 is a chart illustrating data from Example 2.

### DETAILED DESCRIPTION OF PREFERRED EMBODIMENTS

### I. Definitions

For the purposes of this invention, the term “about” in relation to the amounts expressed in weight percent for amounts greater than 2% means that the amount of the component can vary by an amount of +/−1% by weight.

For the purposes of this invention, the term “about” in relation to the amounts expressed in weight percent for amounts less than 2% and greater than 1% means that the amount of the component can vary by an amount of +/−0.2% by weight.

For the purposes of this invention, the term “about” in relation to the amounts expressed in weight percent for amounts less than 1% and greater than 0.5% means that the amount of the component can vary by an amount of +/−0.1% by weight.

For the purposes of this invention, the term “about” in relation to the amounts expressed in milligrams/milliliter (mg/mL) means that the amount of the component can vary by an amount of +/−0.05 mg/mL.

For the purposes of this invention, the term “about” in relation to the amounts of a prescribed dose expressed in micrograms (μg) means that the amount of the component can vary by an amount of +/−5 μg.

For the purposes of this invention, the term “composition” is used in the broad sense to include both single phase compositions and compositions that comprise two or more phases, such as for example compositions comprising a continuous liquid phase and solid particles suspended or dispersed in the liquid phase, or for example a gaseous phase with solid particles suspended, dispersed and/or carried by the gaseous phase, as would occur in an aerosol composition.

The term “pharmaceutical composition” is used herein to include any composition which comprises at least one agent, ingredient, drug, compound, composition, or other substance that may be used on, or administered to, a human or animal for a purpose that includes one or more of therapeutic, pharmaceutical, pharmacological, diagnostic, and prophylactic and immunomodulation.

The term “fine particles” means particles that have of a mean particle diameter up to about 10 microns (10μ).

The term “prescribed dose” refers to the amount of salbutamol, including salbutamol sulfate, intended by the manufacturer of an MDI or by a medical professional to be taken at one specified time.

The term “delivered dose” and its abbreviation “DD” refers to the amount of salbutamol particles contained in the volume of composition that exits the actuator nozzle of an MDI and is available to be drawn into a patient's lungs.

The term “fine particle mass” and its abbreviation “FPM” refers to the dose contained in the volume of composition that exists as a single spray from the actuator nozzle of an MDI, in total mass, that is within a respirable range. The dose that is within the respirable range is measured in vitro to be the sum of the dose delivered at stages 2-7 of an Andersen Cascade Impactor operated at a flow rate of 28.3 L/min.

In the context of a composition containing or providing respirable aggregates, particles, drops, etc., such as compositions described herein, the term “fine particle fraction” or “FPF” refers to the proportion of the delivered material relative to the delivered dose (i.e., the amount that exits the actuator of a delivery device, such as an MDI) that is within a respirable range. The amount of delivered material within the respirable range is measured in vitro as the sum of the material delivered at stages 2-7 of an Andersen Cascade Impactor operated at a flow rate of 28.3 L/min.

“Mass median aerodynamic diameter” or “MMAD” as used herein refers to the aerodynamic diameter of an aerosol below which 50% of the mass of the aerosol consists of particles with an aerodynamic diameter smaller than the MMAD, with the MMAD being calculated according to monograph 601 of the United States Pharmacopeia (“USP).

As used herein, the term “carrier” refers to one or more pharmacologically inert substances which provide a continuous phase in which salbutamol particles (including salbutamol sulfate particles) are suspended and which comprise components that exert a sufficiently high vapor pressure at normal room temperature to propel the particles from the canister of an MDI to a patient upon actuation of the MDI's metering valve. Therefore, the term “carrier” encompasses both a single component and a combination of two or more different components that form the medium in which the salbutamol is suspended or otherwise carried. Thus, the 1234ze(E) component of the carrier of present composition acts at least as a propellent.

The term “respirable” generally refers to particles, aggregates, drops, etc. sized such that they can be inhaled and reach the airways of the lung.

The term “delivered dose uniformity” (DDU) for a pharmaceutical composition means the uniformity as measured by USP 601.

The terms “HFC-134a” and “R134a” means 1,1,1,2-tetrafluoroethane.

The terms “HFO-1234ze(E),” and “1234ze(E)” as used herein each mean trans-1,3,3,3-tetrafluoropropene. Unless otherwise stated, “HFO-1234ze” and “1234ze” mean trans-1,3,3,3-tetrafluoropropene.

The term “salbutamol” as used herein encompasses any and all pharmaceutically acceptable versions of salbutamol, including pharmaceutically acceptable salts of salbutamol (such as salbutamol sulfate) and pharmaceutically acceptable esters of salbutamol.

Reference herein to a group of defined items includes all such defined items, including all such items with suffix designations. Thus for example, a reference herein to “Pharmaceutical Compositions 1-22,” is a specific reference to each of Pharmaceutical Compositions 1A, 1B, 1C, 1D, 1E, 1F, 2A, 2B, 2C, 2D, 2E, 2F, 2G and so on.

### II. The Compositions

The preferred pharmaceutical compositions of the present invention, including each of Pharmaceutical Compositions 1-22, are preferably suspensions of the salbutamol in the carrier comprising the other required components of the composition, including particularly the HFO-1234ze(E) and ethanol.

The preferred pharmaceutical compositions of the present invention, including each of Pharmaceutical Compositions 1-22, are physically stable. The preferred pharmaceutical compositions of the present invention, including each of Pharmaceutical Compositions 1-22, are chemically stable. The preferred pharmaceutical compositions of the present invention, including each of Pharmaceutical Compositions 1-22, are physically stable and chemically stable.

### Concentration of Components

The concentration of the components in the present compositions can generally vary widely within the broad scope of the present invention. The concentration of the salbutamol contained in the compositions of the present invention, including each of Pharmaceutical Compositions 1-6, measured as milligrams per milliliter (mg/mL) is preferably from greater than 2.5 mg/mL, or from greater than about 2.5 mg/mL to less than about 5 mg/mL, or from greater than about 2.5 mg/mL to less than about 4.5 mg/mL, or from greater than 2.5 mg/mL to less than about 4.0 mg/mL. Preferred compositions include those identified in the following Tables 1A and 1 B below, with the following designations in the table having the following meanings: “Comp” means that the composition comprises the identified components; CEO means that the composition consists essentially of the identified components; CO means that the composition consists of the identified components.

TABLE 1A

| | | | | | | Salbutamol |
|---|---|---|---|---|---|---|
| Pharmaceutical | | Components, wt % | | | | sulfate |
| Composition | | | | Oleic | Salbutamol | mg/ml in |
| No. | | 1234ze(E)* | Ethanol* | Acid | sulfate | suspension |
| PC7A | Comp | >95 | >2-<5 | NR | 0.1-<0.5 | NF |
| PC7B | CEO | >95 | >2-<5 | NR | 0.1-<0.5 | NR |
| PC7C | CO | >95 | >2-<5 | NR | 0.1-<0.5 | NR |
| PC7D | Comp | >95 | >2-<5 | NR | 0.1-<0.5 | >2.5 |
| PC7E | CEO | >95 | >2-<5 | NR | 0.1-<0.5 | >2.5 |
| PC7F | CO | >95 | >2-<5 | NR | 0.1-<0.5 | >2.5 |
| PC7G | Comp | >95 | >2-<5 | NR | 0.1-<0.5 | >2.5-<5 |
| PC7H | CEO | >95 | >2-<5 | NR | 0.1-<0.5 | >2.5-<5 |
| PC7I | CO | >95 | >2-<5 | NR | 0.1-<0.5 | >2.5-<5 |
| PC7J | Comp | >95 | >2-<5 | NR | 0.1-<0.5 | >2.5-<4.5 |
| PC7K | CEO | >95 | >2-<5 | NR | 0.1-<0.5 | >2.5-<4.5 |
| PC7L | CO | >95 | >2-<5 | NR | 0.1-<0.5 | >2.5-<4.5 |
| PC7M | Comp | >95 | >2-<5 | NR | 0.1-<0.5 | >2.5-<4 |
| PC7N | CEO | >95 | >2-<5 | NR | 0.1-<0.5 | >2.5-<4 |
| PC7O | CO | >95 | >2-<5 | NR | 0.1-<0.5 | >2.5-<4 |
| PC8A | Comp | >95 | >2-<5 | NR | 0.2-0.4 | NR |
| PC8B | CEO | >95 | >2-<5 | NR | 0.2-0.4 | NR |
| PC8C | CO | >95 | >2-<5 | NR | 0.2-0.4 | NR |
| PC8D | Comp | >95 | >2-<5 | NR | 0.2-0.4 | >2.5 |
| PC8E | CEO | >95 | >2-<5 | NR | 0.2-0.4 | >2.5 |
| PC8F | CO | >95 | >2-<5 | NR | 0.2-0.4 | >2.5 |
| PC8G | Comp | >95 | >2-<5 | NR | 0.2-0.4 | >2.5-<5 |
| PC8H | CEO | >95 | >2-<5 | NR | 0.2-0.4 | >2.5-<5 |
| PC8I | CO | >95 | >2-<5 | NR | 0.2-0.4 | >2.5-<5 |
| PC8J | Comp | >95 | >2-<5 | NR | 0.2-0.4 | >2.5-<4.5 |
| PC8K | CEO | >95 | >2-<5 | NR | 0.2-0.4 | >2.5-<4.5 |
| PC8L | CO | >95 | >2-<5 | NR | 0.2-0.4 | >2.5-<4.5 |
| PC8M | Comp | >95 | >2-<5 | NR | 0.2-0.4 | >2.5-<4 |
| PC8N | CEO | >95 | >2-<5 | NR | 0.2-0.4 | >2.5-<4 |
| PC8O | CO | >95 | >2-<5 | NR | 0.2-0.4 | >2.5-<4 |
| PC9A | Comp | 96-98.5 | >2-<5 | NR | 0.1-<0.5 | NR |
| PC9B | CEO | 96-98.5 | >2-<5 | NR | 0.1-<0.5 | NR |
| PC9C | CO | 96-98.5 | >2-<5 | NR | 0.1-<0.5 | NR |
| PC9D | Comp | 96-98.5 | >2-<5 | NR | 0.1-<0.5 | >2.5 |
| PC9E | CEO | 96-98.5 | >2-<5 | NR | 0.1-<0.5 | >2.5 |
| PC9F | CO | 96-98.5 | >2-<5 | NR | 0.1-<0.5 | >2.5 |
| PC9G | Comp | 96-98.5 | >2-<5 | NR | 0.1-<0.5 | >2.5-<5 |
| PC9H | CEO | 96-98.5 | >2-<5 | NR | 0.1-<0.5 | >2.5-<5 |
| PC9I | CO | 96-98.5 | >2-<5 | NR | 0.1-<0.5 | >2.5-<5 |
| PC9J | Comp | 96-98.5 | >2-<5 | NR | 0.1-<0.5 | >2.5-<4.5 |
| PC9K | CEO | 96-98.5 | >2-<5 | NR | 0.1-<0.5 | >2.5-<4.5 |
| PC9L | CO | 96-98.5 | >2-<5 | NR | 0.1-<0.5 | >2.5-<4.5 |
| PC9M | Comp | 96-98.5 | >2-<5 | NR | 0.1-<0.5 | >2.5-<4 |
| PC9N | CEO | 96-98.5 | >2-<5 | NR | 0.1-<0.5 | >2.5-<4 |
| PC9O | CO | 96-98.5 | >2-<5 | NR | 0.1-<0.5 | >2.5-<4 |
| PC10A | Comp | 96-98.5 | >2-<5 | NR | 0.2-0.4 | NR |
| PC10B | CEO | 96-98.5 | >2-<5 | NR | 0.2-0.4 | NR |
| PC10C | CO | 96-98.5 | >2-<5 | NR | 0.2-0.4 | NF |
| PC10D | Comp | 96-98.5 | >2-<5 | NR | 0.2-0.4 | >2.5 |
| PC10E | CEO | 96-98.5 | >2-<5 | NR | 0.2-0.4 | >2.5 |
| PC10F | CO | 96-98.5 | >2-<5 | NR | 0.2-0.4 | >2.5 |
| PC10G | Comp | 96-98.5 | >2-<5 | NR | 0.2-0.4 | >2.5-<5 |
| PC10H | CEO | 96-98.5 | >2-<5 | NR | 0.2-0.4 | >2.5-<5 |
| PC10I | CO | 96-98.5 | >2-<5 | NR | 0.2-0.4 | >2.5-<5 |
| PC10J | Comp | 96-98.5 | >2-<5 | NR | 0.2-0.4 | >2.5-<4.5 |
| PC10K | CEO | 96-98.5 | >2-<5 | NR | 0.2-0.4 | >2.5-<4.5 |
| PC10L | CO | 96-98.5 | >2-<5 | NR | 0.2-0.4 | >2.5-<4.5 |
| PC10M | Comp | 96-98.5 | >2-<5 | NR | 0.2-0.4 | >2.5-<4 |
| PC10N | CEO | 96-98.5 | >2-<5 | NR | 0.2-0.4 | >2.5-<4 |
| PC10O | CO | 96-98.5 | >2-<5 | NR | 0.2-0.4 | >2.5-<4 |
| PC11A | Comp | >95 | 2.5-4 | NR | 0.1-<0.5 | NR |
| PC11B | CEO | >95 | 2.5-4 | NR | 0.1-<0.5 | NR |
| PC11C | CO | >95 | 2.5-4 | NR | 0.1-<0.5 | NR |
| PC11D | Comp | >95 | 2.5-4 | NR | 0.1-<0.5 | >2.5 |
| PC11E | CEO | >95 | 2.5-4 | NR | 0.1-<0.5 | >2.5 |
| PC11F | CO | >95 | 2.5-4 | NR | 0.1-<0.5 | >2.5 |
| PC11G | Comp | >95 | 2.5-4 | NR | 0.1-<0.5 | >2.5-<5 |
| PC11H | CEO | >95 | 2.5-4 | NR | 0.1-<0.5 | >2.5-<5 |
| PC11I | CO | >95 | 2.5-4 | NR | 0.1-<0.5 | >2.5-<5 |
| PC11J | Comp | >95 | 2.5-4 | NR | 0.1-<0.5 | >2.5-<4.5 |
| PC11K | CEO | >95 | 2.5-4 | NR | 0.1-<0.5 | >2.5-<4.5 |
| PC11L | CO | >95 | 2.5-4 | NR | 0.1-<0.5 | >2.5-<4.5 |
| PC11M | Comp | >95 | 2.5-4 | NR | 0.1-<0.5 | >2.5-<4 |
| PC11N | CEO | >95 | 2.5-4 | NR | 0.1-<0.5 | >2.5-<4 |
| PC11O | CO | >95 | 2.5-4 | NR | 0.1-<0.5 | >2.5-<4 |
| PC12A | Comp | >95 | 2.5-4 | NR | 0.2-0.4 | NR |
| PC12B | CEO | >95 | 2.5-4 | NR | 0.2-0.4 | NR |
| PC12C | CO | >95 | 2.5-4 | NR | 0.2-0.4 | NR |
| PC12D | Comp | >95 | 2.5-4 | NR | 0.2-0.4 | >2.5 |
| PC12E | CEO | >95 | 2.5-4 | NR | 0.2-0.4 | >2.5 |
| PC12F | CO | >95 | 2.5-4 | NR | 0.2-0.4 | >2.5 |
| PC12G | Comp | >95 | 2.5-4 | NR | 0.2-0.4 | >2.5-<5 |
| PC12H | CEO | >95 | 2.5-4 | NR | 0.2-0.4 | >2.5-<5 |
| PC12I | CO | >95 | 2.5-4 | NR | 0.2-0.4 | >2.5-<5 |
| PC12J | Comp | >95 | 2.5-4 | NR | 0.2-0.4 | >2.5-<4.5 |
| PC12K | CEO | >95 | 2.5-4 | NR | 0.2-0.4 | >2.5-<4.5 |
| PC12L | CO | >95 | 2.5-4 | NR | 0.2-0.4 | >2.5-<4.5 |
| PC12M | Comp | >95 | 2.5-4 | NR | 0.2-0.4 | >2.5-<4 |
| PC12N | CEO | >95 | 2.5-4 | NR | 0.2-0.4 | >2.5-<4 |
| PC12O | CO | >95 | 2.5-4 | NR | 0.2-0.4 | >2.5-<4 |
| PC13A | Comp | 96-98.5 | 2.5-4 | NR | 0.1-<0.5 | NR |
| PC13 | CEO | 96-98.5 | 2.5-4 | NR | 0.1-<0.5 | NR |
| PC13C | CO | 96-98.5 | 2.5-4 | NR | 0.1-<0.5 | NR |
| PC13D | Comp | 96-98.5 | 2.5-4 | NR | 0.1-<0.5 | >2.5 |
| PC139E | CEO | 96-98.5 | 2.5-4 | NR | 0.1-<0.5 | >2.5 |
| PC13F | CO | 96-98.5 | 2.5-4 | NR | 0.1-<0.5 | >2.5 |
| PC13G | Comp | 96-98.5 | 2.5-4 | NR | 0.1-<0.5 | >2.5-<5 |
| PC13H | CEO | 96-98.5 | 2.5-4 | NR | 0.1-<0.5 | >2.5-<5 |
| PC13I | CO | 96-98.5 | 2.5-4 | NR | 0.1-<0.5 | >2.5-<5 |
| PC13J | Comp | 96-98.5 | 2.5-4 | NR | 0.1-<0.5 | >2.5-<4.5 |
| PC13K | CEO | 96-98.5 | 2.5-4 | NR | 0.1-<0.5 | >2.5-<4.5 |
| PC13L | CO | 96-98.5 | 2.5-4 | NR | 0.1-<0.5 | >2.5-<4.5 |
| PC13M | Comp | 96-98.5 | 2.5-4 | NR | 0.1-<0.5 | >2.5-<4 |
| PC13N | CEO | 96-98.5 | 2.5-4 | NR | 0.1-<0.5 | >2.5-<4 |
| PC13O | CO | 96-98.5 | 2.5-4 | NR | 0.1-<0.5 | >2.5-<4 |
| PC14A | Comp | 96-98.5 | 2.5-4 | NR | 0.2-0.4 | NR |
| PC14B | CEO | 96-98.5 | 2.5-4 | NR | 0.2-0.4 | NR |
| PC14C | CO | 96-98.5 | 2.5-4 | NR | 0.2-0.4 | NR |
| PC14D | Comp | 96-98.5 | 2.5-4 | NR | 0.2-0.4 | >2.5 |
| PC14E | CEO | 96-98.5 | 2.5-4 | NR | 0.2-0.4 | >2.5 |
| PC14F | CO | 96-98.5 | 2.5-4 | NR | 0.2-0.4 | >2.5 |
| PC14G | Comp | 96-98.5 | 2.5-4 | NR | 0.2-0.4 | >2.5-<5 |
| PC14H | CEO | 96-98.5 | 2.5-4 | NR | 0.2-0.4 | >2.5-<5 |
| PC14I | CO | 96-98.5 | 2.5-4 | NR | 0.2-0.4 | >2.5-<5 |
| PC14J | Comp | 96-98.5 | 2.5-4 | NR | 0.2-0.4 | >2.5-<4.5 |
| PC14K | CEO | 96-98.5 | 2.5-4 | NR | 0.2-0.4 | >2.5-<4.5 |
| PC14L | CO | 96-98.5 | 2.5-4 | NR | 0.2-0.4 | >2.5-<4.5 |
| PC14M | Comp | 96-98.5 | 2.5-4 | NR | 0.2-0.4 | >2.5-<4 |
| PC14N | CEO | 96-98.5 | 2.5-4 | NR | 0.2-0.4 | >2.5-<4 |
| PC14O | CO | 96-98.5 | 2.5-4 | NR | 0.2-0.4 | >2.5-<4 |

*The amounts of 1234ze(E) and ethanol are understood to be preceded in this table by the term “about” as defined above for the relevant amount.

TABLE 1B

| Pharmaceutical | | Components, wt % | | | | Salbutamol |
|---|---|---|---|---|---|---|
| Composition | | | | Oleic | | mg/ml in |
| No. | | 1234ze(E)* | Ethanol* | Acid | Salbutamol | suspension |
| PC15A | Comp | >95 | >2-<5 | NR | 0.1-<0.5 | NR |
| PC15B | CEO | >95 | >2-<5 | NR | 0.1-<0.5 | NR |
| PC15C | CO | >95 | >2-<5 | NR | 0.1-<0.5 | NR |
| PC15D | Comp | >95 | >2-<5 | NR | 0.1-<0.5 | >2.5 |
| PC15E | CEO | >95 | >2-<5 | NR | 0.1-<0.5 | >2.5 |
| PC15F | CO | >95 | >2-<5 | NR | 0.1-<0.5 | >2.5 |
| PC15G | Comp | >95 | >2-<5 | NR | 0.1-<0.5 | >2.5-<5 |
| PC15H | CEO | >95 | >2-<5 | NR | 0.1-<0.5 | >2.5-<5 |
| PC15I | CO | >95 | >2-<5 | NR | 0.1-<0.5 | >2.5-<5 |
| PC15J | Comp | >95 | >2-<5 | NR | 0.1-<0.5 | >2.5-<4.5 |
| PC15K | CEO | >95 | >2-<5 | NR | 0.1-<0.5 | >2.5-<4.5 |
| PC15L | CO | >95 | >2-<5 | NR | 0.1-<0.5 | >2.5-<4.5 |
| PC15M | Comp | >95 | >2-<5 | NR | 0.1-<0.5 | >2.5-<4 |
| PC15N | CEO | >95 | >2-<5 | NR | 0.1-<0.5 | >2.5-<4 |
| PC15O | CO | >95 | >2-<5 | NR | 0.1-<0.5 | >2.5-<4 |
| PC16A | Comp | >95 | >2-<5 | NR | 0.2-0.4 | NR |
| PC16B | CEO | >95 | >2-<5 | NR | 0.2-0.4 | NR |
| PC16C | CO | >95 | >2-<5 | NR | 0.2-0.4 | NR |
| PC16D | Comp | >95 | >2-<5 | NR | 0.2-0.4 | >2.5 |
| PC16E | CEO | >95 | >2-<5 | NR | 0.2-0.4 | >2.5 |
| PC16F | CO | >95 | >2-<5 | NR | 0.2-0.4 | >2.5 |
| PC16G | Comp | >95 | >2-<5 | NR | 0.2-0.4 | >2.5-<5 |
| PC16H | CEO | >95 | >2-<5 | NR | 0.2-0.4 | >2.5-<5 |
| PC16I | CO | >95 | >2-<5 | NR | 0.2-0.4 | >2.5-<5 |
| PC16J | Comp | >95 | >2-<5 | NR | 0.2-0.4 | >2.5-<4.5 |
| PC16K | CEO | >95 | >2-<5 | NR | 0.2-0.4 | >2.5-<4.5 |
| PC16L | CO | >95 | >2-<5 | NR | 0.2-0.4 | >2.5-<4.5 |
| PC16M | Comp | >95 | >2-<5 | NR | 0.2-0.4 | >2.5-<4 |
| PC16N | CEO | >95 | >2-<5 | NR | 0.2-0.4 | >2.5-<4 |
| PC16O | CO | >95 | >2-<5 | NR | 0.2-0.4 | >2.5-<4 |
| PC17A | Comp | 96-98.5 | >2-<5 | NR | 0.1-<0.5 | NR |
| PC17B | CEO | 96-98.5 | >2-<5 | NR | 0.1-<0.5 | NR |
| PC17C | CO | 96-98.5 | >2-<5 | NR | 0.1-<0.5 | NR |
| PC17D | Comp | 96-98.5 | >2-<5 | NR | 0.1-<0.5 | >2.5 |
| PC17E | CEO | 96-98.5 | >2-<5 | NR | 0.1-<0.5 | >2.5 |
| PC17F | CO | 96-98.5 | >2-<5 | NR | 0.1-<0.5 | >2.5 |
| PC17G | Comp | 96-98.5 | >2-<5 | NR | 0.1-<0.5 | >2.5-<5 |
| PC17H | CEO | 96-98.5 | >2-<5 | NR | 0.1-<0.5 | >2.5-<5 |
| PC17I | CO | 96-98.5 | >2-<5 | NR | 0.1-<0.5 | >2.5-<5 |
| PC17J | Comp | 96-98.5 | >2-<5 | NR | 0.1-<0.5 | >2.5-<4.5 |
| PC17K | CEO | 96-98.5 | >2-<5 | NR | 0.1-<0.5 | >2.5-<4.5 |
| PC17L | CO | 96-98.5 | >2-<5 | NR | 0.1-<0.5 | >2.5-<4.5 |
| PC17M | Comp | 96-98.5 | >2-<5 | NR | 0.1-<0.5 | >2.5-<4 |
| PC17N | CEO | 96-98.5 | >2-<5 | NR | 0.1-<0.5 | >2.5-<4 |
| PC17O | CO | 96-98.5 | >2-<5 | NR | 0.1-<0.5 | >2.5-<4 |
| PC18A | Comp | 96-98.5 | >2-<5 | NR | 0.2-0.4 | NR |
| PC18B | CEO | 96-98.5 | >2-<5 | NR | 0.2-0.4 | NR |
| PC18C | CO | 96-98.5 | >2-<5 | NR | 0.2-0.4 | NR |
| PC18D | Comp | 96-98.5 | >2-<5 | NR | 0.2-0.4 | >2.5 |
| PC18E | CEO | 96-98.5 | >2-<5 | NR | 0.2-0.4 | >2.5 |
| PC70F | CO | 96-98.5 | >2-<5 | NR | 0.2-0.4 | >2.5 |
| PC18G | Comp | 96-98.5 | >2-<5 | NR | 0.2-0.4 | >2.5-<5 |
| PC18H | CEO | 96-98.5 | >2-<5 | NR | 0.2-0.4 | >2.5-<5 |
| PC18I | CO | 96-98.5 | >2-<5 | NR | 0.2-0.4 | >2.5-<5 |
| PC18J | Comp | 96-98.5 | >2-<5 | NR | 0.2-0.4 | >2.5-<4.5 |
| PC18K | CEO | 96-98.5 | >2-<5 | NR | 0.2-0.4 | >2.5-<4.5 |
| PC18L | CO | 96-98.5 | >2-<5 | NR | 0.2-0.4 | >2.5-<4.5 |
| PC18M | Comp | 96-98.5 | >2-<5 | NR | 0.2-0.4 | >2.5-<4 |
| PC18N | CEO | 96-98.5 | >2-<5 | NR | 0.2-0.4 | >2.5-<4 |
| PC18O | CO | 96-98.5 | >2-<5 | NR | 0.2-0.4 | >2.5-<4 |
| PC19A | Comp | >95 | 2.5-4 | NR | 0.1-<0.5 | NR |
| PC19B | CEO | >95 | 2.5-4 | NR | 0.1-<0.5 | NR |
| PC19C | CO | >95 | 2.5-4 | NR | 0.1-<0.5 | NR |
| PC19D | Comp | >95 | 2.5-4 | NR | 0.1-<0.5 | >2.5 |
| PC19E | CEO | >95 | 2.5-4 | NR | 0.1-<0.5 | >2.5 |
| PC19F | CO | >95 | 2.5-4 | NR | 0.1-<0.5 | >2.5 |
| PC19G | Comp | >95 | 2.5-4 | NR | 0.1-<0.5 | >2.5-<5 |
| PC19H | CEO | >95 | 2.5-4 | NR | 0.1-<0.5 | >2.5-<5 |
| PC19I | CO | >95 | 2.5-4 | NR | 0.1-<0.5 | >2.5-<5 |
| PC19J | Comp | >95 | 2.5-4 | NR | 0.1-<0.5 | >2.5-<4.5 |
| PC19K | CEO | >95 | 2.5-4 | NR | 0.1-<0.5 | >2.5-<4.5 |
| PC19L | CO | >95 | 2.5-4 | NR | 0.1-<0.5 | >2.5-<4.5 |
| PC19M | Comp | >95 | 2.5-4 | NR | 0.1-<0.5 | >2.5-<4 |
| PC19N | CEO | >95 | 2.5-4 | NR | 0.1-<0.5 | >2.5-<4 |
| PC19O | CO | >95 | 2.5-4 | NR | 0.1-<0.5 | >2.5-<4 |
| PC20A | Comp | >95 | 2.5-4 | NR | 0.2-0.4 | NR |
| PC20B | CEO | >95 | 2.5-4 | NR | 0.2-0.4 | NR |
| PC20C | CO | >95 | 2.5-4 | NR | 0.2-0.4 | NR |
| PC20D | Comp | >95 | 2.5-4 | NR | 0.2-0.4 | >2.5 |
| PC20E | CEO | >95 | 2.5-4 | NR | 0.2-0.4 | >2.5 |
| PC20F | CO | >95 | 2.5-4 | NR | 0.2-0.4 | >2.5 |
| PC20G | Comp | >95 | 2.5-4 | NR | 0.2-0.4 | >2.5-<5 |
| PC20H | CEO | >95 | 2.5-4 | NR | 0.2-0.4 | >2.5-<5 |
| PC20I | CO | >95 | 2.5-4 | NR | 0.2-0.4 | >2.5-<5 |
| PC20J | Comp | >95 | 2.5-4 | NR | 0.2-0.4 | >2.5-<4.5 |
| PC20K | CEO | >95 | 2.5-4 | NR | 0.2-0.4 | >2.5-<4.5 |
| PC20L | CO | >95 | 2.5-4 | NR | 0.2-0.4 | >2.5-<4.5 |
| PC20M | Comp | >95 | 2.5-4 | NR | 0.2-0.4 | >2.5-<4 |
| PC20N | CEO | >95 | 2.5-4 | NR | 0.2-0.4 | >2.5-<4 |
| PC20O | CO | >95 | 2.5-4 | NR | 0.2-0.4 | >2.5-<4 |
| PC21A | Comp | 96-98.5 | 2.5-4 | NR | 0.1-<0.5 | NR |
| PC21 | CEO | 96-98.5 | 2.5-4 | NR | 0.1-<0.5 | NR |
| PC21C | CO | 96-98.5 | 2.5-4 | NR | 0.1-<0.5 | NR |
| PC21D | Comp | 96-98.5 | 2.5-4 | NR | 0.1-<0.5 | >2.5 |
| PC21E | CEO | 96-98.5 | 2.5-4 | NR | 0.1-<0.5 | >2.5 |
| PC21F | CO | 96-98.5 | 2.5-4 | NR | 0.1-<0.5 | >2.5 |
| PC21G | Comp | 96-98.5 | 2.5-4 | NR | 0.1-<0.5 | >2.5-<5 |
| PC21H | CEO | 96-98.5 | 2.5-4 | NR | 0.1-<0.5 | >2.5-<5 |
| PC21I | CO | 96-98.5 | 2.5-4 | NR | 0.1-<0.5 | >2.5-<5 |
| PC21J | Comp | 96-98.5 | 2.5-4 | NR | 0.1-<0.5 | >2.5-<4.5 |
| PC21K | CEO | 96-98.5 | 2.5-4 | NF | 0.1-<0.5 | >2.5-<4.5 |
| PC21L | CO | 96-98.5 | 2.5-4 | NR | 0.1-<0.5 | >2.5-<4.5 |
| PC21M | Comp | 96-98.5 | 2.5-4 | NR | 0.1-<0.5 | >2.5-<4 |
| PC21N | CEO | 96-98.5 | 2.5-4 | NR | 0.1-<0.5 | >2.5-<4 |
| PC21O | CO | 96-98.5 | 2.5-4 | NR | 0.1-<0.5 | >2.5-<4 |
| PC22A | Comp | 96-98.5 | 2.5-4 | NR | 0.2-0.4 | NR |
| PC22B | CEO | 96-98.5 | 2.5-4 | NF | 0.2-0.4 | NR |
| PC22C | CO | 96-98.5 | 2.5-4 | NF | 0.2-0.4 | NR |
| PC22D | Comp | 96-98.5 | 2.5-4 | NR | 0.2-0.4 | >2.5 |
| PC22E | CEO | 96-98.5 | 2.5-4 | NR | 0.2-0.4 | >2.5 |
| PC22F | CO | 96-98.5 | 2.5-4 | NR | 0.2-0.4 | >2.5 |
| PC22G | Comp | 96-98.5 | 2.5-4 | NR | 0.2-0.4 | >2.5-<5 |
| PC22H | CEO | 96-98.5 | 2.5-4 | NR | 0.2-0.4 | >2.5-<5 |
| PC22I | CO | 96-98.5 | 2.5-4 | NR | 0.2-0.4 | >2.5-<5 |
| PC22J | Comp | 96-98.5 | 2.5-4 | NR | 0.2-0.4 | >2.5-<4.5 |
| PC22K | CEO | 96-98.5 | 2.5-4 | NR | 0.2-0.4 | >2.5-<4.5 |
| PC22L | CO | 96-98.5 | 2.5-4 | NR | 0.2-0.4 | >2.5-<4.5 |
| PC22M | Comp | 96-98.5 | 2.5-4 | NR | 0.2-0.4 | >2.5-<4 |
| PC22N | CEO | 96-98.5 | 2.5-4 | NR | 0.2-0.4 | >2.5-<4 |
| PC22O | CO | 96-98.5 | 2.5-4 | NR | 0.2-0.4 | >2.5-<4 |

*The amounts of 1234ze(E) and ethanol are understood to be preceded in this table by the term “about” as defined above for the relevant amount.

For all compositions of the present invention, other than those defined as “consisting of” the designated components, including each of Pharmaceutical Compositions 1-22, additional components or excipients may be present. These components may have various uses and functions, including, but not limited to, facilitating formation of a suspension, stabilizing a suspension, and/or aiding in chemical stabilization of salbutamol or other components.

Preferred excipients include are suitable for inhaled delivery and do not substantially degrade or dissolve in the suspension medium, and preferably are suspension formation aids, suspension stabilizers, salbutamol stabilizers and combinations of these. In particular cases, the excipient is selected from the group consisting of lipids, phospholipids, carbohydrates, amino acids, organic salts, peptides, proteins, alditols, synthetic or natural polymers, surfactant materials and combinations of these.

For all compositions of the present invention, including each of Pharmaceutical Compositions 1-22, the composition comprises a suspension of the indicated salbutamol in the HFO-1234ze(E) and ethanol, and oleic acid when present. In all such compositions, including each of Pharmaceutical Compositions 1-22, the designated salbutamol is preferably in a microparticulate solid form (preferably micronized, but it can also be size-reduced by a multitude of other particle size reduction techniques). As used herein, a suspension of salbutamol may also have a very small amount of solubilized particulate material within the composition. For the present compositions, including each of Pharmaceutical Compositions 1-22, solubilization of salbutamol is generally undesirable. In embodiments, including each of Pharmaceutical Compositions 1-22, there may exists a minimal or nominal solubilization of the salbutamol, but in preferred embodiments including each of Pharmaceutical Compositions 1-22, there is essentially no measurable solubilization of the salbutamol.

In certain preferred forms, the compositions of the present invention, including each of Pharmaceutical Compositions 1-22, have a Global Warming Potential (GWP) of not greater than about 300, more preferably not greater than about 150, not greater than 75, and most preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

### III. Devices and Methods

The present invention includes devices for the delivery by inhalation the composition of the present invention, including each of Pharmaceutical Compositions 1-22. In certain preferred embodiments, the devices of the present invention comprise a container, preferably an aerosol canister, containing a pressurized composition of the present invention, including each of Pharmaceutical Compositions 1-22, and preferably having a metered dose dispensing valve operable between non-dispensing and dispensing positions. The present devices preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

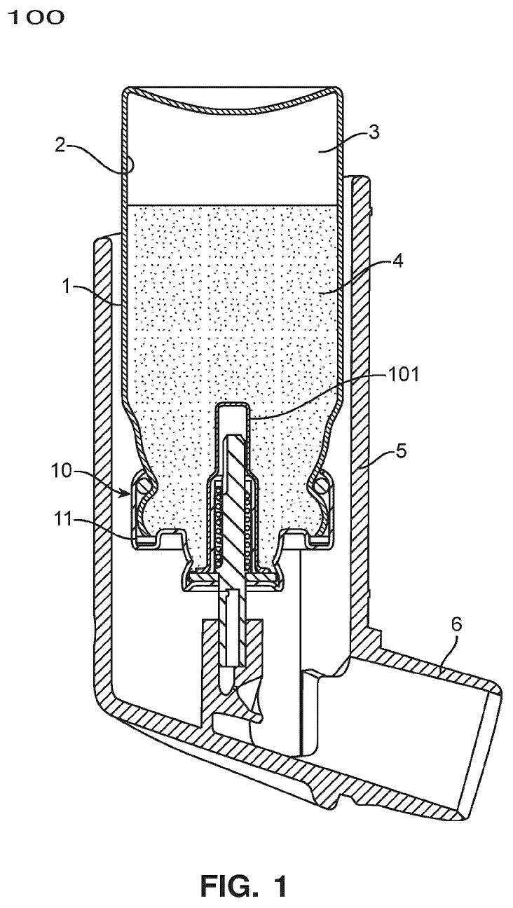

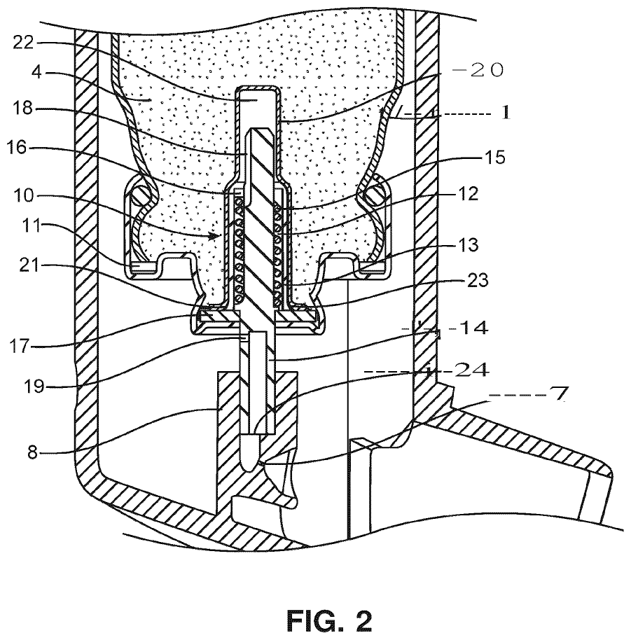

By way of example but not by way of limitation, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol composition 4 is contained.

The valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol composition 4 can pass from the composition chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIG. 1 and FIG. 2, allowing composition to pass from the metering chamber 12 through a side hole 19 in the valve stem and through a stem outlet 24 to an actuator nozzle 7 then out to the patient. When the valve stem 14 is released, composition enters into the valve 10, in particular into the pre-metering chamber 22, through the annular space 21 and thence from the pre-metering chamber through a groove 18 in the valve stem past the tank seal 16 into the metering chamber 12.

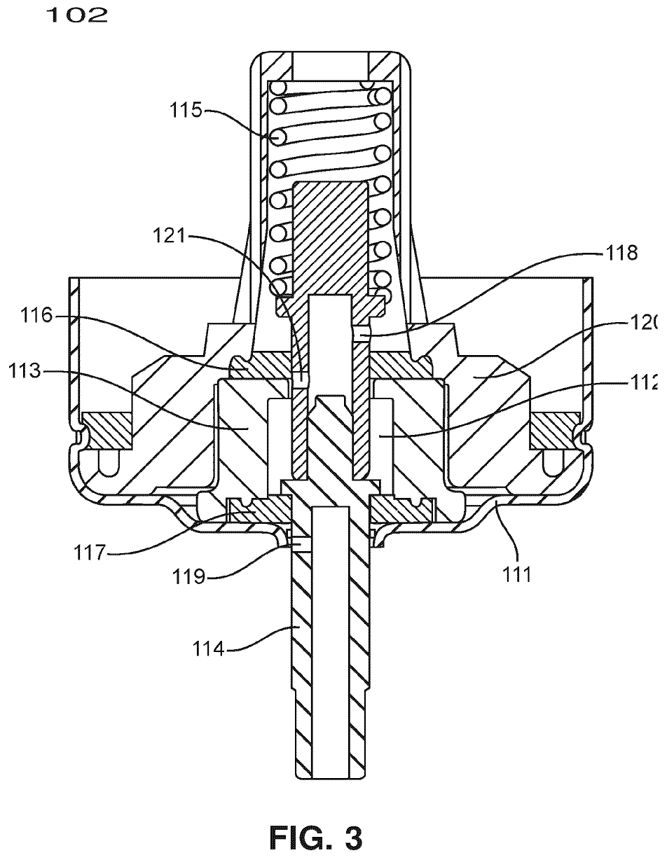

FIG. 3 shows another embodiment of a metered dose aerosol metering valve 102, different from the embodiment shown in FIG. 1 and FIG. 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, composition enters the metering chamber via orifices 121 and 118. The composition's outward path from the metering chamber 112 when a dose is dispensed is via orifice 119.

In certain embodiments the invention device is constructed such that airflow due to patient inhalation is prevented or reduced in the vicinity of the orifice at all times or only during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possesses air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interdispersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle, but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition of the present invention, including each of Pharmaceutical Compositions 1-22, contained in the canister preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention, including each of Pharmaceutical Compositions 1-22, in the canister may be from 1.0 grams (g) to 30.0 g, 2.0 g to 20.0 g, or 5.0 to 10.0 g. The total amount of composition of the present invention, including each of Pharmaceutical Compositions 1-22, is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensure that the amount of each dose remains relatively constant through the life of the inhaler.

The present invention thus provides inhalers, and preferably metered dose inhalers (MDIs) for the treatment of asthma and other chronic obstructive pulmonary diseases and for delivery of pharmaceutical compositions, including each of Pharmaceutical Compositions 1-22, to accessible mucous membranes or intranasally. The present invention thus includes methods for delivering of pharmaceutical compositions, including each of Pharmaceutical Compositions 1-22, for purpose of treating ailments, diseases and similar health related problems of an organism (such as a human or animal) comprising applying a composition of the present invention containing a medicament or other therapeutic component to the organism in need of treatment. In certain preferred embodiments, the step of applying the present composition comprises providing an MDI containing the composition of the present invention, including each of Pharmaceutical Compositions 1-22, and then discharging the present composition from the MDI.

The MDI metering valve size, that is, the size of the metering chamber, can vary within the scope hereof, but may be between 200 microliters (μL or mci) and 400 microliters, between 200 microliters and 360 microliters, between 200 microliters and 340 microliters, between 200 microliters and 320 microliters, or between 200 microliters and 300 microliters.

In certain embodiments, typical compositions of the present disclosure include the salbutamol sulfate in an amount of at least 0.2 milligram per actuation (mg/actuation) (200 microgram (μg) per actuation), or at least 0.25 mg/actuation (250 μg/actuation). In certain embodiments, typical compositions of the present disclosure include the salbutamol sulfate in an amount of less than 0.4 mg/actuation (400 μg/actuation).

In embodiments, typical compositions of the present disclosure include the salbutamol sulfate in an amount of less than 500 μg/actuation, at most 400 μg/actuation, at most 300 μg/actuation. In some preferred embodiments, compositions of the present disclosure include the salbutamol sulfate in an amount of 200 μg/actuation to 400 μg/actuation.

The present invention includes methods of forming pharmaceutical compositions having improved stability, including each of Pharmaceutical Compositions 1-22, comprising forming a carrier comprising not less than 96% by weight and from greater than about 2% by weight to less than 5% by weight of ethanol, suspending in said carrier said salbutamol, preferably wherein said suspension has at least about great than 2.5 and less than about 4 mg/mL of salbutamol.

The present invention includes methods of forming pharmaceutical compositions having improved stability, including each of Pharmaceutical Compositions 1-22, comprising forming a carrier comprising not less than 96% by weight and from greater than about 2% by weight to less than 5% by weight of ethanol, suspending in said carrier said salbutamol sulfate, preferably wherein said suspension has at least about great than 2.5 and less than about 4 mg/mL of salbutamol sulfate.

The present invention includes methods of forming pharmaceutical compositions having improved stability, including each of Pharmaceutical Compositions 1-22, comprising forming a carrier comprising not less than 96% by weight and from greater than about 2% by weight to less than 5% by weight of ethanol, suspending in said carrier an API comprising, consisting essentially of, or consisting of from 0.25 wt. % to about 0.5 wt. % salbutamol.

### EXAMPLES

### Comparative Example 1: Delivered Dose and Particle Size Measurement for Suspension of Salbutamol Sulfate Suspension in 100% HFO-1234e(E) and in a Mixture of HFO-1234ze(E) and Ethanol with Nominal Dose of 100 μg Per Actuation

Salbutamol sulfate (API) was suspended in HFC-1234ze(E) and a mixture of HFC-1234ze(E) and ethanol in amounts as indicated in Example 3 of WO2023/039103A1 and as reported in Table C1 below. Each suspension was formed with 1.91 mg/mL of salbutamol sulfate to provide a nominal dose of 100 μg/actuation. Each suspension was filled into FEP coated canisters, crimped with a 63-pL valve and tested with a KINDEVA actuator having an exit orifice diameter of 0.4 mm for testing. The fine particle mass (FPM), fine particle fraction (FPF), and delivered dose were measured for each suspension. Results as reported are shown in Table C1 below.

TABLE ExC1

| Example No. | ExC1A | ExC1B | ExC1C | ExC1E |
|---|---|---|---|---|
| Components | | | | |
| HFC-1234ze(E), | 100 | 98 | 95 | 85 |
| wt. % carrier | | | | |
| Ethanol, wt. % | 0 | 2 | 5 | 15 |
| carrier | | | | |
| Total Carrier | 100 | 100 | 100 | 100 |
| API, mg/mL | 1.91 | 1.91 | 1.91 | 1.91 |
| Total | 100 | 100 | 100 | 100 |
| Spray | | | | |
| Properties | | | | |
| FPM, | 45.3 | 45.9 | 36.8 | 18.5 |
| μg/actuation | | | | |
| FPF (%) | 57.5 | 48.5 | 39.1 | 19.6 |
| DD, μg/actuation | 63.8 | 77.7 | 82.7 | 85.8 |
| DDU (Pass/Fail) | Fail | Pass | Pass | Pass |

In general, the prescribed amount of API is achieved with two actuations of the MDI, that is, the nominal prescribed dose per use is 200 μg.

### Example 1: Delivered Dose and Particle Size Measurement for Suspension of Salbutamol Sulfate Suspension in Mixtures of HFO-1234ze(E) and Ethanol with Nominal API Dose of 200 μg per Actuation

Salbutamol sulfate (API) was suspended in HFC-1234ze(E) and mixtures of HFC-1234ze(E) and ethanol in amounts as indicated in Table E1 below. Each suspension was formed with about 3.8 mg/mL of salbutamol sulfate to provide a nominal dose of about 200 μg/actuation. Each suspension was filled into FEP coated canisters, crimped with a 63-pL valve, and tested with a commercially available actuator. The fine particle mass (FPM), fine particle fraction (FPF), and delivered dose were measured for each suspension. Results as reported are shown in Table E1 below.

TABLE E1

| Example No. | E1A | E1B |
|---|---|---|
| Components | | |
| HFC-1234ze(E), | 98 | 95 |
| wt. % carrier | | |
| Ethanol, wt. % | 2 | 5 |
| carrier | | |
| Total Carrier | 100 | 100 |
| API, mg/mL | 3.82 | 3.82 |
| Total | 100 | 100 |
| Spray Properties | | |
| FPM, μg/actuation | 104.9 | 89.4 |
| FPF (%) | 54.7 | 49.0 |
| DD, μg/actuation | 191.6 | 182.3 |
| DDU (Pass/Fail) | Pass | Pass |

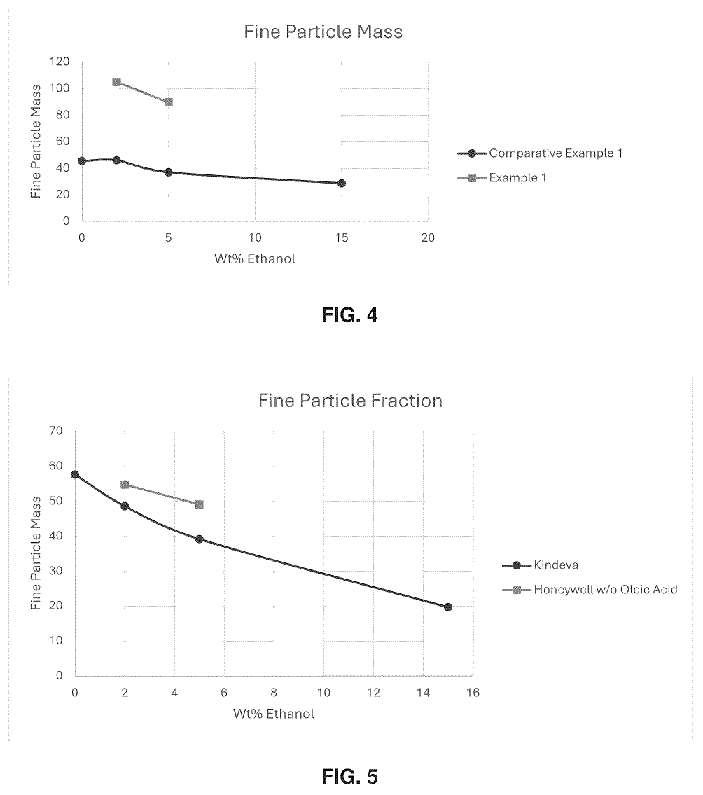

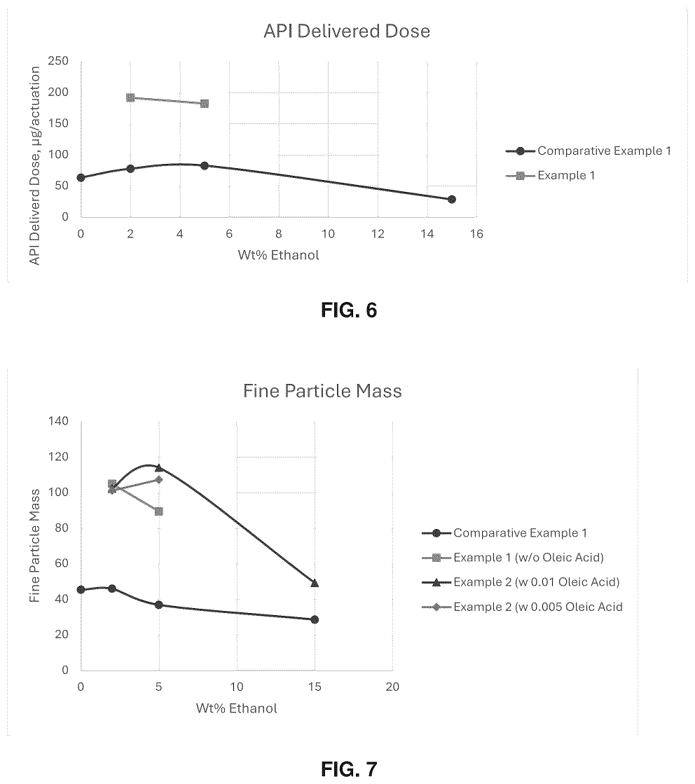

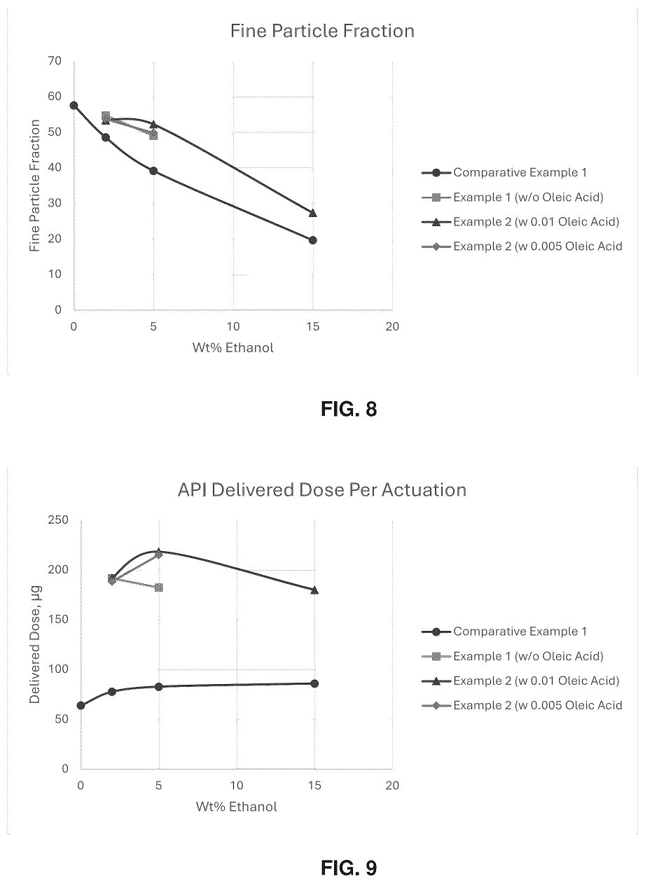

As is seen from test result reported in Table Ex1 above (an illustrated in the FIGS. 4-6), applicants have found that compositions, systems and methods according to the preferred aspects of the present invention produce important but unexpected results and advantages. Among these unexpected advantages is the ability to provide a composition which forms a stable suspension while at the same time provides an APR delivered dose that is greater than 150 micrograms per actuation, which is substantially and unexpectedly much more than two times (>2×) higher than the value achieved by Comparative Example 1. Furthermore, the fine particle fraction achieved according to the present invention is unexpectedly improved by a significant amount compared to the results from Comparative Example 1. The fine particle dose per actuation (FPM) is also substantially and unexpectedly substantially higher than the values achieved according to Comparative Example 1. These unexpected advantages are illustrated in the following charts comparing the results of Comparative Example 1 and Example 1.

### Example 2: Delivered Dose and Particle Size Measurement for Suspension of Salbutamol Sulfate Suspension in Mixtures of HFO-1234ze(E),Ethanol and Oleic Acid With Nominal API Dose of 200 μg per actuation

Salbutamol sulfate (API) was suspended in mixtures of HFC-1234ze(E), ethanol and oleic acid in amounts as indicated in Table E2 below. Each suspension was formed with about 3.8 mg/mL of salbutamol sulfate to provide a nominal API dose of about 200 μg/actuation. Each suspension was filled into FEP coated canisters, crimped with a 63-pL valve, and tested with a commercially available actuator. The fine particle mass (FPM), fine particle fraction (FPF), and delivered dose (ex-act) were measured for each suspension. Results are shown in Table E2 below.

TABLE E2

| Example No. | E2A | E2B | E2C | E2D | E2E |
|---|---|---|---|---|---|
| Components | | | | | |
| HFC-1234ze(E), | 97.99 | 97.995 | 94.99 | 94.995 | 84.99 |
| wt. % carrier | | | | | |
| Ethanol, wt. % | 2 | 2 | 5 | 5 | 15 |
| carrier | | | | | |
| Oleic Acid | 0.01 | 0.005 | 0.01 | 0.005 | 0.01 |
| Total Carrier | 100 | 100 | 100 | 100 | 100 |
| API, mg/mL | 3.82 | 3.82 | 3.82 | 3.82 | 3.82 |
| Total | 100 | 100 | 100 | 100 | 100 |
| Spray | | | | | |
| Properties | | | | | |
| FPM, | 102.2 | 101.1 | 113.9 | 107.2 | 49.2 |
| μg/actuation | | | | | |
| FPF (%) | 53.3 | 53.7 | 52.2 | 49.8 | 27.3 |
| DD, μg/actuation | 191.9 | 188.4 | 218.3 | 215.1 | 179.9 |
| DDU (Pass/Fail) | Pass | Pass | Pass | Pass | Pass |

As is seen from test result reported in Table E2 above, applicants have found that compositions according to preferred aspects of the present invention produce not only the important and unexpected results and advantages described in connection with Example 1 above, but also additional unexpected and important advantages. Among the additional unexpected advantages associated with the use of oleic acid in the present compositions occurs for oleic acid amounts of greater than 0.005 wt. % (and preferably greater than 0.005% to about 0.001%) and for composition with ethanol concentrations of from greater than 2% to less than about 6%. Under these conditions applicants have unexpectedly found that deterioration in FPM and API Delivered Dose that occurs as the concentration of ethanol increases above 2% for the data for compositions without oleic, the preferred oleic acid and ethanol amounts of the preferred embodiments of this example produce an unexpected reversal of this trend and produces an unexpectedly high maximum for FPM and API Delivered Dose. This unexpected result is illustrated in the following charts which include the data from Comparative Example 1, Example 1 and Example 2.

What is claimed is:

1. A pharmaceutical composition comprising:
a. fine particles of salbutamol;
b. from greater than 95% by weight to less than about 98% by weight of HFO-1234ze(E); and
c. from greater than 2% by weight to less than 5% by weight of ethanol.

2. The pharmaceutical composition of claim 1 consisting essentially of:
a. said fine particles of salbutamol;
b. said HFO-1234ze(E); and
c. said ethanol.

3. The pharmaceutical composition of claim 2 further comprising from greater than 0.005% by weight to less than 0.015% by weight of oleic acid.

4. The pharmaceutical composition of claim 1 wherein said fine particles of salbutamol comprise fine particles of salbutamol sulfate.

5. The pharmaceutical composition of claim 1 wherein said fine particles of salbutamol consist essentially of fine particles of salbutamol sulfate.

6. The pharmaceutical composition of claim 1 wherein said fine particles of salbutamol consist of fine particles of salbutamol sulfate.

7. The pharmaceutical composition of claim 1 wherein said salbutamol is present in an amount of from about 2.5 mg/mL to less than about 4.5 mg/mL.

8. The pharmaceutical composition of claim 7 wherein said salbutamol is present in an amount of from about 2.5 mg/mL to about 3.5 mg/mL.

9. An aerosol comprising the pharmaceutical composition of claim 1.

10. The aerosol of claim 9 wherein the amount of said salbutamol in said aerosol is from about greater than 150 micrograms to about 300 micrograms.

11. The pharmaceutical composition of claim 1 wherein said HFO-1234ze(E) is present in an amount off from greater than 96% by weight to less than about 98% by weight and said ethanol is present in an amount of from greater than 2% by weight to less than 4% by weight of ethanol.

12. A pharmaceutical composition comprising:
a. from about 2.5 mg/mL to less than about 4.5 mg/mL of fine particles salbutamol sulfate;
b. a carrier in which said salbutamol sulfate is suspended, said carrier comprising: (i) from about 95% by weight to less than 99% by weight of HFO-1234ze(E); and (ii) from greater than 1% by weight to about 5% by weight of ethanol.

13. The pharmaceutical composition of claim 12 consisting essentially of:
said salbutamol sulfate particles;
said HFO-1234ze(E); and
said ethanol.

14. The pharmaceutical composition of claim 12 further comprising from greater than 0.005% by weight to less than 0.015% by weight of oleic acid.

15. The pharmaceutical composition of claim 14 further comprising from greater than 0.005% by weight to about 0.01% by weight of oleic acid.

16. A pharmaceutical composition in the form of an aerosol comprising:
a. fine particles of salbutamol sulfate; and
b. a carrier for said fine particulate salbutamol sulfate, said carrier comprising: (i) from about 94% by weight to less than 99% by weight of HFO-1234ze(E); (ii) from greater than 1% by weight to about 6% by weight of ethanol, wherein the amount of said salbutamol sulfate in said aerosol is from about greater than 150 micrograms to about 300 micrograms.

17. The pharmaceutical composition of claim 15 further comprising oleic acid.

18. A method for delivering a prescribed dose of salbutamol sulfate comprising:
a. providing in an MDI comprising an actuator and a pharmaceutical composition comprising: (1) a carrier comprising from greater than about 95% by weight to less than 99% by weight of HFO-1234ze(E); and (ii) from greater than 1% by weight to about 5% by weight of ethanol; and (2) fine particles of salbutamol sulfate; and
b. actuating said MDI once to produce a single aerosol spray of said salbutamol sulfate, wherein said single spray contains the prescribed dose of said salbutamol sulfate.

19. The method of claim 17 wherein said single spray contains said prescribed dose of said salbutamol sulfate, wherein said prescribed dose is an amount of from about 150 micrograms to about 300 micrograms.

20. The method of claim 17 wherein said aerosol spray consists essentially of:
a. said salbutamol sulfate;
b. said HFO-1234ze(E);
c. said ethanol; and
d. from greater than 0.005% by weight to less than 0.015% by weight of oleic acid.
